(19) United States
(12) Patent Application Publication
(10) Pub. No.: US 2007/0155006 A1
(43) Pub. Date: Jul. 5, 2007

(54) Photobioreactor

(76) Inventor: Alexander Levin, Binyamina (IL)

Correspondence Address: Alexander Levin, 5A Shvil Hachalav St., Binyamina, 30500, omitted

(21) Appl. No.: 11/642,061

(22) Filed: Dec. 20, 2006

Related U.S. Application Data
(60) Provisional application No. 60/754,950, filed on Dec. 30, 2005.

(51) Int. Cl. C12M 1/00; A01G 7/00

(52) U.S. Cl. 435/292.1; 47/1.4

(57) ABSTRACT

An invention proposes a construction of a photobioreactor, which is based on application of parallel sets of multi-level troughs intended for flowing a microalgae suspension, these troughs are irradiated therewith by the sun light. The troughs are arranged in each set one above the other horizontally or with a small inclination to the horizontal plane. The width of the gaps between the neighboring sets of the troughs is significantly larger than the width of the troughs themselves; at the same time, the sum of the widths of each troughs' set is significantly higher (up to several times) than the width of the above-mentioned gap. Optical elements, which reflect and disperse the light, are positioned between the neighboring sets of the troughs. In addition, multi-tubular elements with headers may be used in the proposed construction instead of the troughs.

### CROSS-REFERENCE TO RELATED APPLICATIONS

This application claims the benefit of U.S. Provisional Application No. 60/754,950, filed Dec. 30, 2005, entitled “PHOTOBIOREACTOR” (Alexander Levin) which is incorporated herein by reference in its entirety.

### FIELD OF THE INVENTION

This invention relates to the area of bioreactors intended to cultivate microalgae.

### BACKGROUND OF THE INVENTION

Mass cultivation of microalgae has a great potential for modern agriculture, biochemistry and pharmaceutics.

Algal species: Spirulina, Dunaliella and others present important sources of vitamins, proteins, unsaturated fats, organic compounds of iron and other microelements. The most common forms of microalgae cultivation photobioreactors are ponds or open channels (raceways).

There are some technical problems connected with application of such systems:

a) It is very difficult to achieve uniform light distribution within the photobioreactor constructed as ponds or channels. The depth of a pond should be in the range of 15÷30 cm. It determines, in turn, relatively low final microalgae concentration in nutritious solution and high cost of harvesting microalgae biomass;
b) It is necessary to perform mixing the nutritious solution in order to prevent cell sinking, and, besides, to remove from the nutritious solution the generated oxygen, which inhibits photosynthesis process;
c) It is necessary to provide an adequate amount of CO₂ which required for performance of photosynthesis process; this CO₂ is supplied as a rule from the ambient air by dissolution in the nutritious solution;
d) It is necessary to maintain optimum ranges of daily and nightly temperatures of the nutritious solutions.

There is a significant number of patents and patent applications which are devoted to solve a part of the above-mentioned problems; however, these patents and patent application do not provide sufficiently effective and cheap solutions of the described problems.

According to U.S. Pat. No. 395,317 plant cells are grown continuously in a tubular transparent plastic structure through which water containing nutrients and carbon dioxide is passed. A suspension of plant cells in the water grow on exposure to light and can be harvested as a food material.

U.S. Pat. No. 4,084,346 to E. Stengel and C. J. Socder: “Method and arrangement for optimally supplying autotrophic organisms with CO₂ nutrient” (1978) describes a system of channels intended for algae growing, there are discharge means installed in these channels which introduce CO₂ gas into the microalgae suspension.

U.S. Pat. No. 4,676,956 discloses an apparatus for photosynthesis includes a unique arrangement for supplying light and CO.sub.2-containing air to a reaction chamber in a sure and stable manner. The reaction chamber is irradiated for photosynthesis from the inside and/or outside thereof in an intermittent mode. CO.sub.2-containing air is routed from a CO.sub.2 source to a rotatable disc which is positioned in a bottom portion of the apparatus. Part of the CO.sub.2-containing air is ejected sideways from the disc to cause it into rotation, while the rest of the air is ejected upwardly into the reaction chamber. The reaction chamber is partitioned into a plurality of compartments which are sequentially supplied with the CO.sub.2-containing air in accordance with the rotation of the disc.

U.S. Pat. No. 3,650,068 to C. Meyer and M. Rebellen: “Process of growing algae” (1972) describes a regulation unit for adjustment of a solution concentration in a system of microalgae growing.

U.S. Pat. No. 4,217,728 to H. Shimamatsu and Y. Tominage: “Apparatus for cultivating algae” (1978) proposes a flow rectifying means intended to be installed in a rectangular basin corners.

U.S. Pat. No. 4,868,123 to X. Benson et al.: “Apparatus for the intensive controlled production of microorganisms by photosynthesis” (1989) describes a general system of microalgae growing, which includes columns for introducing CO₂ into the microalgae suspension and a degassing means for eliminating the oxygen from this suspension.

U.S. Pat. No. 4,724,214 to Kei Mori: “Apparatus for photosynthesis” (1988) proposes a photosynthetic reaction bath with a number of photoradiators in the form of narrow upright tubes.

U.S. Pat. No. 4,952,511 to R. Radmer describes a photobioreactor for the cultivation of photosynthetic microorganisms, which comprises a tank, one or more light compartments extending into the tank and one or more high intensity lamps whose light is directed into the light compartments. Each light compartment has at least one transparent wall and a means for distributing light from the lamp substantially uniformly across the transparent wall.

U.S. Pat. No. 5,104,803 to J. Delente proposes a photobioreactor for the cultivation of photosynthetic microorganisms having at least one light bank substantially totally immersible in the liquid microbial culture contained in the photobioreactor so that substantially all of the emitted light is absorbed in the culture. The light bank comprises a plurality of light tubes in substantially close proximity to each other.

According to U.S. Pat. No. 5,137,828, the production of biomass, such as algae, is carried out in a substantially transparent tube wound on an upstanding core structure. The exterior surface of the tube is exposed to natural light and the tube and/or the core is adapted to encourage light penetration into the tube in the region of contact between the tube and the core structure.

U.S. Pat. No. 5,151,347 to J. Delente et al. describes an apparatus for the controlled production of microorganisms by photosynthesis in a closed photobioreactor. The closed photobioreactor contain a photosynthetic culture in a substantially sealed environment and provides a system for re-circulating the reactant gas through the culture.

U.S. Pat. No. 5,162,051 to S. Hoeksema describes a photobioreactor for the cultivation of photosynthetic microorganisms, wherein a plurality of baffles are mounted in the photobioreactor tank forming hollow cavities which enable the insertion of light sources through openings in the tank wall.

U.S. Pat. No. 5,242,827 describes an apparatus for the automatic, continuous cleaning of the pipe of a solar receptor of a photobioreactor also having a carbonator associated with the solar receptor.

U.S. Pat. No. 5,541,056 to Mark E. Huntley et al.: “Method of control of microorganism growth process” (1996) describes a design of a growing chamber; this chamber has such size and a form that this provides required turbulent flow regime with maintaining a predetermined range of ratios between the scale of the chamber and the scale of turbulent eddies of the suspension flow.

U.S. Pat. No. 5,846,816 discloses a bioreactor for biomass production having (i) a substantially transparent chamber, the chamber being suitable for containing biomass in a liquid phase, and having a base portion, an upper portion and a number of sidewalls between the base portion and the upper portion, the sidewalls being configured so as to diverge from the base portion towards the upper portion; and (ii) a circulating means for circulating the contents of the chamber to create a motive force in the liquid phase to ensure continual mixing of all of the biomass and a semi-continuous exposure of the biomass to a light source.

U.S. Pat. No. 6,174,720 to A. Muller-Feuga describes a bioreactor apparatus for culture of living matter in a liquid medium includes a plurality of tubes connected at one end to a primary manifold section and at their other end to a secondary manifold section such that a flow of liquid containing the living matter can be established within the manifolds and tubes.

U.S. Pat. No. 6,348,347 relates to a culture device of a domed shape, a conical shape, or a cylindrical shape of a closed type used for culture of microalgae, and a gas discharge device set so as to be movable in the culture device. The culture device is basically composed of a transparent inside member (a semispherical dome, a conical peripheral wall, or a cylindrical peripheral wall), a transparent outside member, and a bottom portion connecting the lower ends of the two members, a cylindrical opening portion is provided at the top part of the outside member, and a gas introducing member and a discharging member of a culture solution are provided in the bottom portion. The gas discharge device is basically composed of two opposed rectangular base plates, a bubble guide member, and a discharge nozzle. The culture solution can be agitated without mechanical agitation and the culture can be carried out in high concentrations.

According to U.S. Pat. No. 6,370,815 photosynthetic organisms are grown in a tube having a gas inlet at one end and a gas outlet at the other. The tube containing a rotor having vanes adapted to wipe the inside surface, the tube being disposed at an angle to the horizontal in a bath containing liquid, the gas inlet being lowermost.

U.S. Pat. No. 6,492,149 proposes a method of improving the yield of a photobioreactor of the continuously operating recirculation type. This method includes application of transparent or respecting particles introduced into the reaction medium, which particles are of a density that is substantially equal to that of the reaction medium, thereby adjusting absorbance of the microorganism culture to optimum levels.

U.S. Pat. No. 6,602,703 discloses a photobioreactor for cultivating a photosynthetic organism. This photobioreactor provides innovative features that allow an easy cleaning of the light source. The photobioreactor has a container for containing a liquid culture medium for cultivating photosynthetic organisms; light-emitting tubes are mounted within the container. The photobioreactor also has cleaning devices mounted within the container for cleaning the outer surface of the light-emitting tubes and actuators for actuating the cleaning devices.

U.S. Pat. No. 6,603,069 to J. Muhs et al. proposes an adaptive full spectrum solar energy system having at least one hybrid solar concentrator, at least one hybrid luminaire, at least one hybrid photobioreactor, and a light distribution system operably connected to each hybrid solar concentrator, each hybrid luminaire, and each hybrid photobioreactor. A lighting control system operates each component.

U.S. Pat. No. 6,602,703 to F. Dutil describes a photobioreactor for cultivating a photosynthetic organism. The photobioreactor has a container for containing a liquid culture medium for cultivating photosynthetic organisms, light-emitting tubes mounted within the container. The photobioreactor has cleaning devices mounted within the container for cleaning the outer surface of the light-emitting tubes and actuators for actuating the cleaning devices.

U.S. Pat. No. 6,509,188 to W. Trosh et al. describes a photobioreactor which has a reactor chamber that is made of light-transparent material and that has an increased surface area.

U.S. Pat. No. 6,815,204 relates to a method of improving the transfer in an annular biological reaction chamber defined by coaxial inner and outer walls and in which there flows a liquid reaction medium containing a culture of microorganisms or of cells from vegetable or animal macroorganisms in suspension. At least one of the walls is an exchange wall enabling gaseous or liquid matter to be transferred or allowing light to pass through. The reaction medium is subjected to a turbulent primary flow that is helical and that under the action of centrifugal force creates rotary secondary vortices so as to encourage renewal of the culture in the vicinity of the exchange wall.

U.S. patent application No. 20030073231 to F. Dutil describes a photobioreactor for cultivating a photosynthetic organism. The photobioreactor has a container for containing a liquid culture medium for cultivating photosynthetic organisms, light-emitting tubes mounted within the container. The photobioreactor also has cleaning devices mounted within the container for cleaning the outer surface of the light-emitting tubes and actuators for actuating the cleaning devices.

U.S. patent application No. 20040048364 to W. Trosch proposes o a bioreactor for cultivating microorganisms, as well as a method for its production. The bioreactor comprises two identically constructed base elements that are constructed in trough shape and consist of a bottom part and four side parts arranged on a bottom part. The base element consists of a light permeable material. The identically constructed base elements are arranged on each other so as to exactly cover each other. A flow guide device is arranged inside the identically constructed base elements.

U.S. patent application No. 200302286 to I. Burbidge et al. is related to a photobioreactor which comprises an upstanding core structure; a plurality of substantially transparent tubes supportable by the core structure; flow means for causing a synthesis mixture to flow through each of the transparent tubes; and withdrawal means for withdrawing a biomass synthesis product from the mixture. The plurality of transparent tubes is helically wound in parallel.

U.S. patent application No. 20030059932 to J. Craigie et al. describes a photobioreactor for mass production of algae in a liquid pool, comprising a vessel including first and second generally parallel walls. The vessel is adapted to receive a liquid pool. A plurality of hollow tubes extends from the first wall to the second wall for receiving a light source. The hollow tubes are adapted to be immersed in the liquid pool such that the light source can illuminate the liquid pool.

U.S. patent application No. 20050255584 to J. Broneske et al. proposes a bioreactor for culturing microorganisms which has a reactor vessel, a plurality of gas-introduction tubes and a gas-introduction system for introducing gas into a culture medium in the gas-introduction tubes via injectors, wherein the gas-introduction tubes are connected by their respective lower end, in the vertical direction, to the reactor vessel and by their opposite upper end to the upper end of an upright vessel which is likewise connected by its lower end to the reactor vessel, and at the upper end of the upright vessel an expansion vessel is arranged.

Reviews of technical problems related to designs of industrial photobioreactors are presented in the articles: James C. Ogbonna, Hideo Tanaka “Industrial-size photobioreactors” CHEMTECH 1997, 27(7), 43-49 and O. Pulz “PHOTOBIOREACTORS: PRODUCTION SYSTEM FOR PHOTOTROPHIC MICROORGANISMS” Springer-Verlag, 2001.

### BRIEF SUMMARY OF THE INVENTION

The proposed construction of a photobioreactor is based on application of parallel sets of troughs intended for flowing microalgae culture when these troughs are radiated by the sun light. In each set, the troughs are arranged one above the other horizontally or with small inclination to the horizontal plane.

The width of the gaps between the neighboring sets of the troughs is significantly larger than the width of the troughs themselves; however, the sum of the widths of each set of the troughs is significantly higher than the width of the gap between the neighboring sets of the troughs.

Optical elements, which reflect and disperse the light penetrating into the gaps between the neighboring sets of the troughs, are positioned in these gaps; the optical elements serve for illumination of the troughs situated especially at the middle and lower levels of the sets.

Each trough may be constructed from relatively short profiled units and in such a way it is possible to build the troughs of a great length.

In addition, the bottoms of the profiled units can be provided with recesses or transverse low partitions which ensure required average depth of the microalgae suspension in the troughs.

There is a system of feeding pipes with nozzles delivering the microalgae suspension to the troughs. The broth with microalgae is flowing from the troughs into a collecting troughs and thereupon this suspension is accumulated in a tank. The suspension is supplied again from the tank by a pumping means into the feeding pipes and thereafter—to the troughs.

There are two main variants of the troughs' construction. In the first one, each trough is installed on two parallel ropes tightened between a pair of vertical posts at a same level, these posts are arranged at a certain distance one from the other. If the troughs have sufficient rigidity, they may be installed at their ends immediately on the vertical posts without application of the ropes. In this case, the vertical posts are designed as vertical ladders, each rung of the ladder-wise post serves for fastening the trough.

Profiled units with lateral shoulders are installed on the parallel tightened ropes by clamps in such a manner, that the ends of the neighboring profiled units are overlapped and the section of the profiled unit positioned higher overlaps the section of the other profiled unit positioned lower. In addition, the adjacent edges of the profiled units may be joined by welding or, in the case, when these edges are provided with an internal or external flanging, these edges can be joined mechanically with application of sealing gaskets.

In addition, the profiled units can be provided with recesses at their bottoms, these recesses ensure required average depth of the layer of the microalgae suspension in the troughs, which have a certain inclination because deflection of the supporting ropes. In another version, the bottom of the trough is provided with low transverse partitions.

There is a set of the parallel ropes tightened between the pair of the vertical posts at some levels, and each couple of the parallel ropes situated at a certain level serves for installation one or more troughs assembled from the profiled units.

In the second version each trough is constructed from two parallel rods installed at the same level on a pair of the vertical posts, these rods are mutually joined by a set of arc-wise (with convexity downwards) transverse braces. A flexible strip with the width which is somewhat greater, than the length of these arc-wise transverse braces, is installed on a couple of rods joined by the set of the transverse braces in such a way that the lateral edges of the flexible strip are fastened on these rods. The arc-wise transverse braces ensure recesses' formation on the bottom of the troughs and therefore—generation of sufficiently high average depth of microalgae suspension in the troughs.

The entire set of the vertical rows of the troughs with the optical elements positioned between these vertical rows can be placed in a greenhouse construction which prevents ingress of the dust into the microalgae suspension.

Multi-tubular elements with headers may be used in the proposed construction instead of the troughs. The tubes in the multi-tubular elements are made from a transparent polymer. The headers are provided with inlet and outlet connections.

In addition, the headers can be provided with shoulders which serve for fastening these multi-tubular elements on the vertical posts. In this case the whole system for microalgae growing must be provided with a column (or columns) for oxygen stripping and introducing CO₂ in the microalgae suspension.

### BRIEF DESCRIPTION OF THE SEVERAL VIEWS OF DRAWINGS

The present invention will be understood more fully from the detailed description given below and from the accompanying drawings of the preferred embodiment of the invention in which:

FIG. 1 is a top plan view of the field with the photobioreactor and the accompanied units.

FIG. 2 illustrates a side-elevation view of the multi-level troughs in one set.

FIG. 3 is a vertical cross-section of several sets of the multi-level troughs and the optical elements.

FIG. 4a is a top plan view of a profiled unit which serves for the troughs' construction.

FIGS. 4b and 4c are cross-sections of the aforementioned profiled unit.

FIG. 4d is a side-elevation view of the aforementioned profiled unit.

FIG. 5a illustrates a top view of the frame constructed from two parallel rods and the arc-wise transverse braces.

FIG. 5b is a cross-section of the frame constructed from the rods and the arc-wise transverse braces.

FIG. 6a and FIG. 6b are a side-elevation view and cross-section A-A of the multi-tubular element.

FIG. 7 shows the cross-section of the oblong transparent tube which serves for alternative construction of the multi-tubular element.

### DETAILED DESCRIPTION OF THE INVENTION

A photobioreactor for microalgae growing is disclosed with some specific details of troughs' construction. However, it will be apparent to one skilled in the art, that the present invention may be practiced without these specific details.

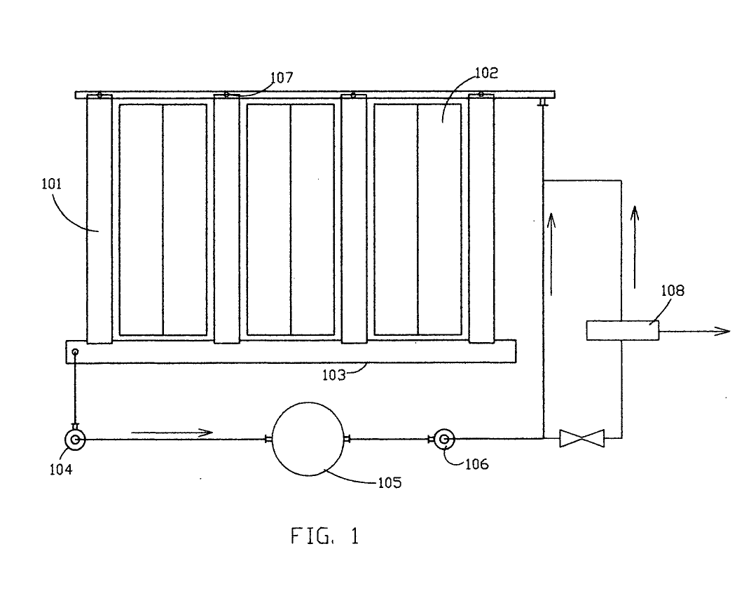

With reference to FIG. 1 a preferred embodiment of a system of microalgae growing including a proposed photobioreactor is illustrated. This system comprises: troughs 101, reflecting optical elements 102, a collecting trough 103, a first pumping means 104, tank 105 for microalgae suspension storage, a second pumping means 106, a feeding pipe 107.

In addition, the system may include other units, which are not shown in this drawing, for example, a column for oxygen stripping and saturation of the microalgae suspension with CO₂.

A side elevation view of the multi-level troughs installed in their one set is presented in FIG. 2. It comprises a pair of vertical posts 201 with circular grooves 202; ropes 203 are tightened between these posts in such a manner, that at a specific level there is a couple of the tightened parallel ropes which are placed apart at a distance corresponding the width of the shoulders of profiled units 204.

Each couple of ropes 203 situated at a same level serves as a support for fastening the overlapped profiled units 204; these units form troughs. Feeding pipes 205 serve for delivery of the microalgae suspension into troughs 206, this suspension is discharged into a collecting trough 207. Troughs 206 can be constructed from profiled units 208 made from a transparent polymer. In another version the lower surface of these profiled units has high reflecting characteristics. The adjacent edges of two profiled units are overlapped, or joined by welding. In the case, when the edges are provided with internal or external flanging, these adjacent edges can be mutually joined with application of sealing gaskets. The entire system of troughs with the optical elements 208 is placed in a greenhouse with glazing 209.

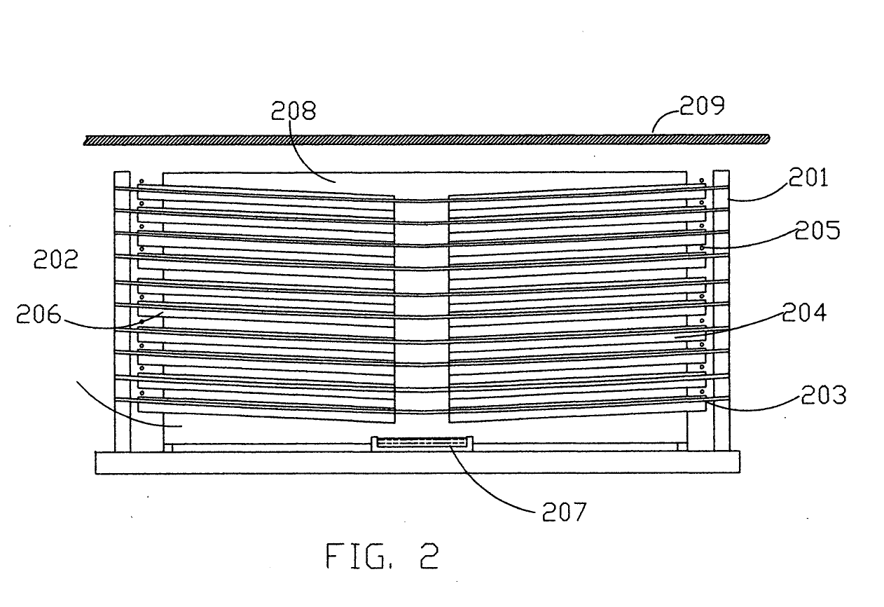

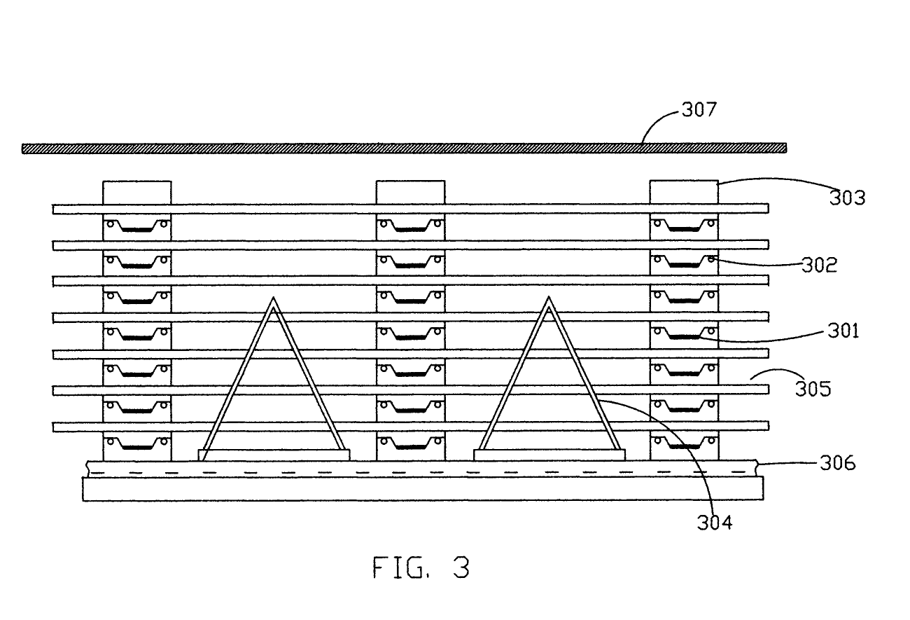

FIG. 3 shows the vertical cross-section of a preferred embodiment of the sets of multi-level troughs. Multi-level troughs 301 are mounted on supporting ropes 302, these ropes are fastened on vertical posts 303; reflecting optical elements 304 are installed in the gaps between the neighboring sets of the multi-level troughs 301.

Feeding pipes 305 provide the microalgae suspension into troughs 301. Glazing 306 of a greenhouse covers the area of the sets of the multi-level troughs with the reflecting optical elements 304.

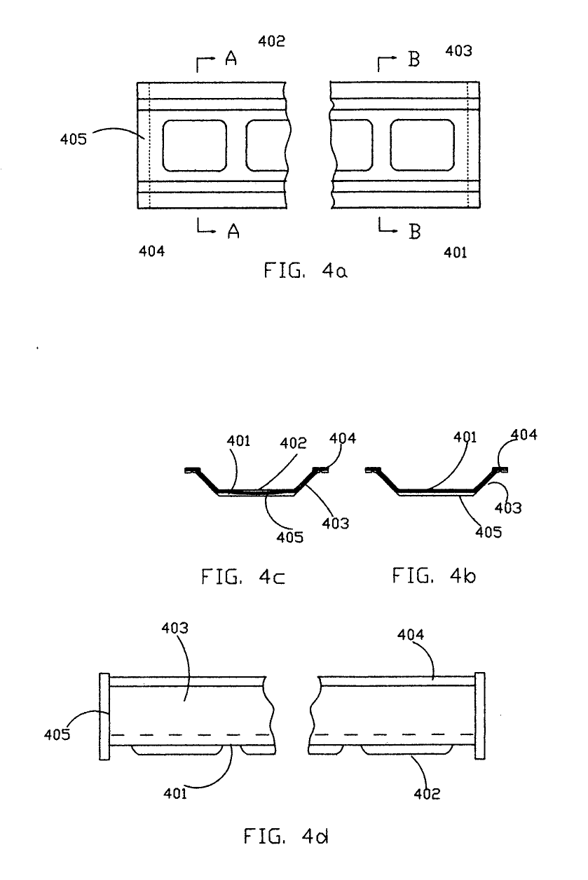

FIG. 4a, 4b, 4c show a top plan view, cross-sections and a side-elevation view of a profiled unit which serves for the troughs' construction.

This profiled unit has following elements: bottom 401 with recesses 402; lateral walls 403; shoulders 404; an external flanging 405.

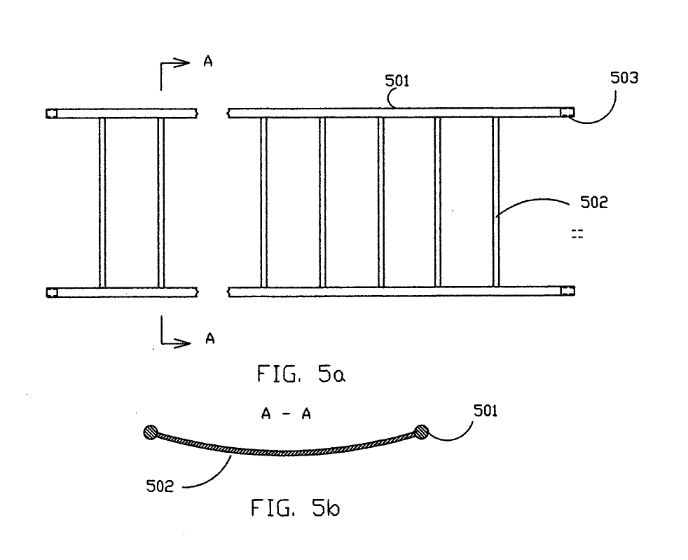

FIGS. 5a and 5b illustrate a side-elevation view and a cross-section of the frame constructed from two parallel rods 501 and transverse braces 502. The ends of the rods are provided with threads 503 which serve for installation of these frames on vertical posts.

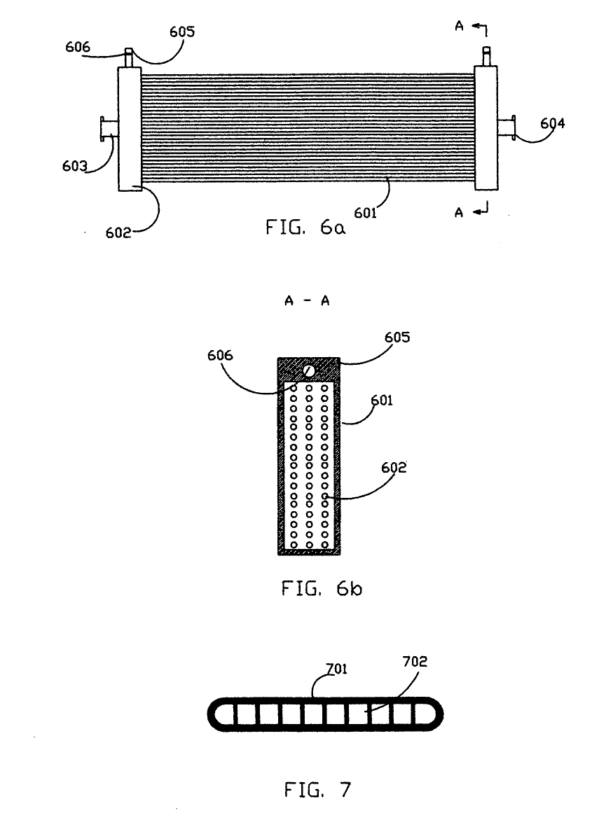

FIGS. 6a and 6b shows a side-elevation view and cross-section A-A of the multi-tubular element. It comprises transparent tubes 601; headers 602; an inlet connection 603; an outlet connection 604; shoulders 605 with openings 606 serve for installation of these multi-tubular elements on vertical posts.

FIG. 7 shows a cross-section of tube 701 incorporated in construction of the multi-tubular element, this cross-section is elongated in the horizontal direction. In addition, tube 701 is provided with vertical partitions 702.

What is claimed is:

1. A photobioreactor intended for mass cultivation of microalgae, said photobioreactor comprising:
parallel sets of multi-level troughs intended for flowing a microalgae suspension; said troughs are arranged in each said set one above the other horizontally; the width of the gaps between the neighboring sets of said multi-level troughs is significantly larger than the width of said troughs themselves; at the same time the sum of the widths of each said multi-level troughs' set is significantly higher (up to several times) than the width of said gap between said neighboring sets;
optical elements which reflect and disperse the light, said optical elements are positioned between said neighboring sets of said troughs, said optical elements serve for illumination of said troughs situated at the middle and lower levels of said troughs' sets by the light, which penetrates into the gaps between said neighboring troughs' sets;
feeding pipes which ensure delivery of said microalgae suspension into said troughs;
collecting troughs which serve for removal of said microalgae suspension from said troughs;
a greenhouse construction, which serves for arrangement of said troughs, said optical elements, said collecting troughs and said feeding pipes in its internal space.

2. A photobioreactor intended for mass cultivation of microalgae as recited in claim 1, wherein said troughs are arranged in each said set one above the other with a small inclination to the horizontal plane.

3. A photobioreactor intended for mass cultivation of microalgae as recited in claim 1, wherein each said trough is constructed from a number of relatively short profiled units, each said profiled unit is provided with lateral shoulders and the adjacent sections of said profiled units are overlapped;
each said set of said multi-level troughs is carried by a pair of vertical posts with circular grooves; ropes are tightened between said pair of said vertical posts in such a manner, that there is a couple of said tightened parallel ropes at a specific level, which are placed apart at a distance corresponding the width of said shoulders of said profiled units; each said couple of said ropes situated at a same level serves as a support for fastening said overlapped profiled units which form said troughs.

4. A photobioreactor intended for mass cultivation of microalgae as recited in claim 1, wherein said profiled units are made preferably from a transparent polymer.

5. A photobioreactor intended for mass cultivation of microalgae as recited in claim 1, wherein said profiled units are made preferably from a material with high reflecting characteristics of their external lower surface.

6. A photobioreactor intended for mass cultivation of microalgae as recited in claim 3, wherein the adjacent edges of said profiled units are joined by welding.

7. A photobioreactor intended for mass cultivation of microalgae as recited in claim 3, wherein the adjacent edges of said profiled units are provided with an internal or external flanging; in this case said adjacent edges are mutually joined with application of sealing gaskets.

8. A photobioreactor intended for mass cultivation of microalgae as recited in claim 3, wherein said profiled units are provided with recesses at their bottoms, said recesses ensure required average depth of the layer of the microalgae suspension in said troughs, which have a certain inclination as the result of the deflection of said ropes.

9. A photobioreactor intended for mass cultivation of microalgae as recited in claim 1, wherein said profiled units are provided with transversal low partitions at their bottoms, said transversal low partitions ensure required average depth of the layer of the microalgae suspension in said troughs which have a certain inclination as the result of the deflection of said ropes.

10. A photobioreactor intended for mass cultivation of microalgae as recited in claim 1, wherein each said trough is constructed from two parallel rods installed at the same level on a pair of vertical posts, said rods are mutually joined by a set of arc-wise connectors with convexity directed downward, and there is a flexible strip from a polymer film with the width, which is somewhat greater than the length of said arc-wise connectors; said flexible strip is installed on a couple of said rods in such a way that its lateral edges are fastened on said rods; said arc-wise connectors ensure recesses' formation on the bottom of the troughs and therefore—generation of sufficiently high average depth of microalgae suspension in said troughs.

11. A photobioreactor intended for mass cultivation of microalgae as recited in claim 1, wherein a multi-tubular element (or multi-tubular elements) is used instead of each set of said multi-level troughs, each said multi-tubular elements comprises plurality of parallel tubes from a transparent polymer; the ends of said tubes are joined with two headers; said headers are provided with an inlet connection and an outlet connection.

12. A photobioreactor intended for mass cultivation of microalgae as recited in claim 11, wherein said multi-tubular elements are provided with shoulders which serve for installation of said multi-tubular elements on said vertical posts.
